(19) United States
(12) Patent Application Publication
(10) Pub. No.: US 2005/0010175 A1
(43) Pub. Date: Jan. 13, 2005

(54) Piston assembly for syringe

(76) Inventors: Daniel E. Beedon, Elmhurst, IL (US); Erin L. Lundtveit, Wadsworth, IL (US); Edwin Chim, Vernon Hills, IL (US); Craig L. Sandford, Buffalo Grove, IL (US); Derek Walsh, Wonder Lake, IL (US); Jennifer C. Ikeda, Grayslake, IL (US); Carol C. Johns, Antioch, IL (US); Catherine Tong Nguyen, Glenview, IL (US); James H. Rose, Antioch, IL (US); Atif M. Yardimci, Northbrook, IL (US)

Correspondence Address: BAXTER INTERNATIONAL INC., DF2-2E, One Baxter Parkway, Deerfield, IL, 60015-4633, US

(21) Appl. No.: 10/788,818

(22) Filed: Feb. 27, 2004

Related U.S. Application Data
(60) Provisional application No. 60/450,543, filed on Feb. 27, 2003.

(51) Int. Cl. A61M 5/315

(52) U.S. Cl. 604/218

(57) ABSTRACT

A piston assembly (10) for a flowable materials container having a barrel (14) defining a fluid containing chamber (16). The barrel (14) is made of a body of a cyclic olefin containing polymer or a bridged polycyclic olefin containing polymer. An elastomeric piston (24) is slidably attached to the barrel (14) and provides a seal of the chamber. The piston (24) has a parylene coating on an outer surface thereof to reduce the necessary break away force. A plunger (22) is also provided and has a second mating member (42) removably connecting the plunger to a first mating member (44) of the piston. The first mating member (44) has a series of threads having a major diameter and a minor diameter, and the second mating member (42) has a series of threads having a major diameter and a minor diameter. The major and minor diameters of the second mating members (42) is appreciably smaller than the major and minor diameters of the other of the first mating member (44) to provide ease of connection.

This application claims the benefit of U.S. Provisional Application 60/450,543 filed Feb. 27, 2003.

### TECHNICAL FIELD

The present invention relates generally to a piston assembly for a polymeric syringe, and more specifically, to a coated piston and an engagement means for a piston and plunger rod assembly.

### Background

In the past, syringe bodies were typically manufactured of glass. Recently, however, syringe bodies have been manufactured from polymeric resins. Glass syringe bodies have certain disadvantages when compared to polymeric syringe bodies; glass syringe bodies are more costly to produce and caused difficulties during the manufacturing process if the glass chips, cracks or breaks. The broken glass particles would not only become hazards to workers and manufacturing equipment, but could also become sealed within the glass syringe body causing a potential health hazard to a downstream patient.

U.S. Pat. No. 6,065,270 (the '270 patent), issued to Reinhard et al. and assigned to Schott Glaswerke of Germany, describes a method of producing a prefilled, sterile syringe body from a cyclic olefin copolymer (COC) resin. The syringe body of the '270 patent comprises a barrel having a rear end which is open and an outlet end with a head molded thereon and designed to accommodate an injection element, a plunger stopper for insertion into the rear end of the barrel to seal it, and an element for sealing the outlet luer tip. The method of manufacturing the syringe body includes the steps of: (1) forming the syringe body by injection molding a material into a core in a cavity of an injection mold, the mold having shape and preset inside dimensions; (2) opening the mold and removing the formed syringe body, said body having an initial temperature; (3) sealing one end of the barrel of the plastic syringe body; (4) siliconizing an inside wall surface of the barrel of the plastic syringe body immediately after the body is formed and while the body remains substantially at said initial temperature; (5) filling the plastic syringe body through the other end of the barrel of the plastic syringe body; and (6) sealing the other end of the barrel of the plastic syringe body, wherein the method is carried out in a controlled environment within a single continuous manufacturing line. According to the method of the '270 patent, the sterilization step is applied to the filled and completely sealed ready-to-use syringe body. Historically, sterilization of finished syringe components (barrel, plunger, and tip cap) has been conducted using ethylene oxide, moist-heat or gamma irradiation.

U.S. patent application No. 2002/0139008 (the '008 application), published Oct. 3, 2002 also describes a method of producing pre-filled COC syringe in an in-line manufacturing process. In contrast to the '270 patent the process of manufacture may best be described as an asceptic filling process. In the asceptic filling process, the various components are sterilized individually and then brought into a sterile environment through an isolator. In this process, the various components may be sterilized by different means before being transferred into the sterile environment. Once inside the sterile environment, the components may be assembled into a sterile pre-filled syringe that is then transferred out of the sterile environment. After transfer additional packaging steps may be undertaken including insertion and connection of a plunger rod as well as labeling of the syringe and placing in an environmental packaging for handling and shipping.

As explained above, during the syringe assembly, whether it is composed of a glass syringe body or a polymeric syringe body, requires a piston component. In many instances the piston is frequently manufactured of an elastomeric material. The elastomeric material deforms to provide a sterile seal against the inner diameter of the syringe body. The piston is also critical to the operation of syringes, in the aspiration and dispensing of medical fluids. Therefore, it is preferred that the piston provide numerous functional features as it should: (1) be capable of providing a sterile barrier for the contents of the syringe during the anticipated transport and storage of the syringe, (2) remain lubricious enough during storage such that it may be initially activated without excessive force, sometimes referred to as breakaway force, and then slide easily to provide control to the rate and amount of medical fluid being ejected from the syringe; (3) not leach undesirable extractives from the material comprising the piston into the medical solution; (3) supply a vapor barrier to prevent water loss which could modify the concentrations of the solutions in the syringe; and (4) be capable of being sterilized by methods suitable for commercial production of medical devices. To assist in satisfying the surface lubricity requirement above, prior art devices have employed such methods as coating the piston with a PTFE Teflon coating, or a silicone coating and/or coating the interior of the syringe barrel with a silicone coating. However the PTFE Teflon coating adds extra costs and the silicone lubricant in some instances does not appear to adequately satisfies a many of the above-noted requirements.

Also as stated above, to lower the costs of manufacture the assembly of the components should be done in a high throughput assembly fashion. Therefore; the piston assembly must be capable of being fully assembled efficiently and correctly to maintain an appropriate seal during assembly and transport and yet be ergonomically acceptable to the end user. Typically, the piston and the plunger rod have mating thread assemblies to secure the piston to the plunger rod. In one embodiment, the plunger rod has a series of male threads, and the piston has a series of mating female threads. Should it be desired to attach the plunger rod to the piston during assembly, plunger rod is threaded into the threads on the piston to provide an attachment of these components. Assembly of the piston assembly should be accomplished by automated rod insertion equipment.

Automated threading of the plunger rod onto the piston may prove to be difficult and frequently does not allow full insertion of the male threads of the plunger rod in the piston. Moreover, the rod may prematurely engage the piston and spin the piston before the plunger rod is fully seated within the piston. This may result in the breaking of the sterile barrier, and/or in syringes having piston assemblies with plunger rods that are not fully secured to the piston. Accordingly, an improved mating engagement performed by automated rod insertion equipment that allows for ease of secure connection between the piston and the plunger rod is desired.

Additionally, various seal configurations have been employed on the piston to obtain an adequate seal with the syringe barrel. While a positive seal with the barrel is required, if the interference between the piston and the inner surface of the syringe barrel is too great, this will result in undesirably high activation or breakaway forces. As such, an improved seal design, accounting for material, geometry and coating parameters is desired.

### SUMMARY OF THE INVENTION

The present invention provides a piston assembly for a fluid container. According to one aspect of the present invention, the piston assembly provides acceptable breakaway and operational forces, is capable of being sterilized by methods suitable for commercial production of medical devices, and provides a sterile seal during transport and storage.

According to yet an additional aspect of the present invention, the piston is coated with a parylene coating. The parylene coated piston is provided for use within a syringe barrel that is produced from cyclic olefin containing polymers or bridged polycyclic hydrocarbon containing polymers (referred to as “COC”s) which are e-beam sterilized.

According to another aspect of the present invention, the piston has seal geometry comprising a plurality of annular ribs or lobes. The annular lobes assist the piston in providing a sterile seal with the syringe barrel without requiring undesirably excessive forces during breakaway or operation. The annular lobes also assist in providing such a seal over an extended period of storage time.

According to another aspect of the present invention, the piston assembly comprises a piston and a plunger rod. The plunger rod has a first mating member which engages a second mating member of the piston to removably connect the plunger rod to the piston. The first mating member of the plunger rod has a series of threads having a major diameter and a minor diameter, and the second mating member has a series of threads having a major diameter and a minor diameter. The major and minor diameters of one of the first and second mating members are appreciably smaller than the major and minor diameters of the other of the first and second mating members to assist in reducing the connection force of the piston and the plunger rod, and to reduce the chance of the seal being compromised.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a cross-sectional side elevation view of a syringe of the present invention;

FIG. 2 is a side sectional view of a piston which may include one embodiment of a coating of the present invention;

FIG. 3 is a cross-sectional side elevation view of one embodiment of a piston of the present invention;

FIG. 4 is a cross-sectional side elevation view of another embodiment of a piston of the present invention; and,

FIG. 5 is a cross-sectional side elevation view of another embodiment of a piston of the present invention, and

FIG. 6 is a side elevational view of a male threaded end of a plunger of the present invention.

### DETAILED DESCRIPTION OF THE INVENTION

While this invention is susceptible of embodiments in many different forms, there are shown in the drawings and will herein be described in detail, preferred embodiments of the invention with the understanding that the present disclosures are to be considered as exemplifications of the principles of the invention and are not intended to limit the broad aspects of the invention to the embodiments illustrated.

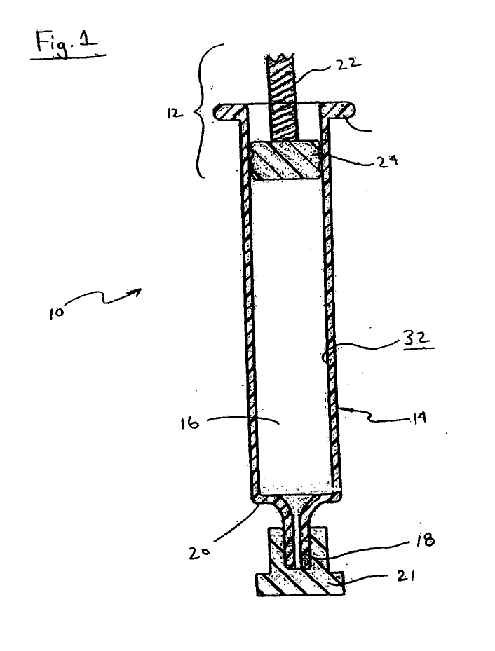

Referring now in detail to the Figures, and initially to FIG. 1, there is shown a syringe 10 having a piston assembly 12 constructed in accordance with the teachings of the present invention. The syringe 10 has a syringe barrel 14 defining a fluid chamber 16 and an elongated luer or tip 18 projecting from a distal end 20 of the syringe barrel 14. Alternatively, the distal end 20 of the syringe barrel 14 may be adapted for receiving an injection needle or the like. The piston assembly 12 is provided in the barrel 14 of the syringe 10 and provides a closure member for the syringe barrel 14. The piston assembly 12 generally comprises a plunger rod 22 and a piston 24 with the plunger rod preferable removably connected to the piston. The piston assembly 12 activates a flow of a fluid substance outwardly from the chamber 16 through the tip 18. The tip 18 of the syringe is typically equipped with a tip cap 21. Such syringes 10 are commonly used in medical applications.

Syringes 10 can be produced from glass or any suitable polymer. In a preferred embodiment of the present invention, the syringes 10 are produced from cyclic olefin containing polymers or bridged polycyclic hydrocarbon containing polymers. These polymers, in some instances, shall be collectively referred to as COCs.

The use of COC-based syringe bodies overcome many of the drawbacks associated with the use of glass syringe bodies. The biggest drawbacks of glass syringe bodies are in connection with the handling of the glass syringes. For instance, the glass syringes are often chipped, cracked, or broken during the manufacturing process. Glass particles may become trapped within the syringe bodies and subsequently sealed within the syringe barrel with the medical solution. This could be hazardous to a patient injected with the medical solution. Additionally, the glass particles could become a manufacturing hazard by causing injury to plant personnel or damage to expensive manufacturing equipment.

Suitable COC polymers include homopolymers, copolymers and terpolymers obtained from cyclic olefin monomers and/or bridged polycyclic hydrocarbons as defined below.

Suitable cyclic olefin monomers are monocyclic compounds having from 5 to about 10 carbons in the ring. The cyclic olefins can be selected from the group consisting of substituted and unsubstituted cyclopentene, cyclopentadiene, cyclohexene, cyclohexadiene, cycloheptene, cycloheptadiene, cyclooctene, cyclooctadiene. Suitable substituents include lower alkyl, acrylate derivatives and the like.

Suitable bridged polycyclic hydrocarbon monomers have two or more rings and more preferably contain at least 7 carbons. The rings can be substituted or unsubstituted. Suitable substitutes include lower alkyl, aryl, aralkyl, vinyl, allyloxy, (meth) acryloxy and the like. The bridged polycyclic hydrocarbons are selected from the group consisting of those disclosed in the below incorporated patents and patent applications and in a most preferred form of the invention is norbornene.

Suitable homopolymer and copolymers of cyclic olefins and bridged polycyclic hydrocarbons and blends thereof can be found in U.S. Pat. Nos. 5,218,049, 5,854,349, 5,863,986, 5,795,945, 5,792,824; EP 0 291,208, EP 0 283,164, EP 0 497,567 which are incorporated in their entirety herein by reference and made a part hereof. These homopolymers, copolymers and polymer blends may have a glass transition temperature of greater than 50° C., more preferably from about 70° C. to about 180° C., a density greater than 0.910 g/cc and more preferably from 0.910 g/cc to about 1.3 g/cc and most preferably from 0.980 g/cc to about 1.3 g/cc and have from at least about 20 mole % of a cyclic aliphatic or a bridged polycyclic in the backbone of the polymer more preferably from about 30-65 mole % and most preferably from about 30-60 mole %.

Suitable comonomers for copolymers and terpolymers of the COCs include α-olefins having from 2-10 carbons, aromatic hydrocarbons, other cyclic olefins and bridged polycyclic hydrocarbons.

The presently preferred COC is a norbornene and ethylene copolymer. These norbornene copolymers are described in detail in U.S. Pat. Nos. 5,783,273, 5,744,664, 5,854,349, and 5,863,986. The norborene ethylene copolymers preferably have from at least about 20 mole percent norbornene monomer and more preferably from about 20 mole percent to about 75 mole percent and most preferably from about 30 mole percent to about 60 mole percent norbornene monomer or any combination or subcombination of ranges therein. The norbornene ethylene copolymer should have a glass transition temperature of from about 70-180° C., more preferably from 70-130° C. The heat deflection temperature at 0.45 Mpa should be from about 70° C. to about 200° C., more preferably from about 75° C. to about 150° C. and most preferably from about 76° C. to about 149° C. Also, in a preferred form of the invention, the COC is capable of withstanding, without significant heat distortion, sterilization by an autoclave process at 121° C. Suitable copolymers are sold by Ticona under the tradename TOPAS under grades 6013, 6015 and 8007 (not autoclavable).

Other suitable COCs are sold by Nippon Zeon under the tradename ZEONEX and ZEONOR, by Daikyo Gomu Seiko under the tradename CZ resin, and by Mitsui Petrochemical Company under the tradename APEL.

It may also be desirable to have pendant groups associated with the COCs. The pendant groups are for compatibilizing the COCs with more polar polymers including amine, amide, imide, ester, carboxylic acid and other polar functional groups. Suitable pendant groups include aromatic hydrocarbons, carbon dioxide, monoethylenically unsaturated hydrocarbons, acrylonitriles, vinyl ethers, vinyl esters, vinylamides, vinyl ketones, vinyl halides, epoxides, cyclic esters and cyclic ethers. The monethylencially unsaturated hydrocarbons include alkyl acrylates, and aryl acrylates. The cyclic ester includes maleic anhydride.

Polymer blends containing COCs have also been found to be suitable for fabricating syringe bodies 14. Suitable two-component blends of the present invention include as a first component a COC in an amount from about 1% to about 99% by weight of the blend, more preferably from about 30% to about 99%, and most preferably from about 35% to about 99% percent by weight of the blend, or any combination or subcombination or ranges therein. In a preferred form of the invention the first component has a glass transition temperature of from about 70° C. to about 130° C. and more preferably from about 70-110° C.

The blends further include a second component in an amount by weight of the blend of about 99% to about 1%, more preferably from about 70% to about 1% and most preferably from about 65% to about 1%. The second component is selected from the group consisting of homopolymers and copolymers of ethylene, propylene, butene, hexene, octene, nonene, decene and styrene. In a preferred form of the invention the second component is an ethylene and α-olefin copolymer where the α-olefin has from 3-10 carbons, and more preferably from 4-8 carbons. Most preferably the ethylene and α-olefin copolymers are obtained using a metallocene catalyst or a single site catalyst. Suitable catalyst systems, among others, are those disclosed in U.S. Pat. Nos. 5,783,638 and 5,272,236. Suitable ethylene and α-olefin copolymers include those sold by Dow Chemical Company under the AFFINITY and ENGAGE tradenames, those sold by Exxon under the EXACT tradename and those sold by Phillips Chemical Company under the tradename MARLEX.

Suitable three-component blends include as a third component a COC selected from those COCs described above and different from the first component. In a preferred form of the invention the second COC will have a glass transition temperature of higher than about 120° C. when the first COC has a glass transition temperature lower than about 120° C. In a preferred form of the invention, the third component is present in an amount by weight of from about 10-90% by weight of the blend and the first and second components should be present in a ratio of from about 2:1 to about 1:2 respectively of the first component to the second component.

In a preferred three-component blend, a second norbornene and ethylene copolymer is added to the two component norbornene-ethylene/ethylene 4-8 carbon α-olefin blend. The second norbornene ethylene copolymer should have a norbornene monomer content of 30 mole percent or greater and more preferably from about 35-75 mole percent and a glass transition temperature of higher than 120° C. when the first component has a glass transition temperature of lower than 120° C.

Referring particularly to FIG. 1, to assist in increasing the lubricity of the piston 24 (i.e., reduce break away and operational forces during operation of the syringe), a lubricant has typically been applied to the inner surface 32 of the syringe barrel 14. Specifically, in prior art devices the inner surface 32 of the syringe barrel 14 and/or piston 24 are siliconized prior to assembly of the piston assembly 12 in the syringe 10. It has been found that in COC syringes which have barrels which have been sterilized by irradiation, siliconizing of the barrel and/or piston does not provide the necessary lubricity particularly in regards to minimizing breakaway force. The inventors believe that the irradiation may excite the molecules at the surface 32 of the barrel 14 and promote the formation of microscopic bonds with the elastomeric material of the piston 24 during the shelf life of the syringe 10. These bonds cause unacceptable breakaway forces.

Accordingly, in the present invention, instead of applying silicone to the piston 24, a parylene coating is applied to piston 24. The surface of the barrel 14 may or may not receive a layer of silicone oil. U.S. Pat. No. 6,270,872, hereby incorporated by reference herein, describes the chemical structure of parylene. The parylene coating provides numerous advantages for the piston assembly 12 over previous coatings such as silicone oil and PTFE Teflon.

Parylene is applied at room temperature with gas phase polymer deposition equipment that allows control of both coating rate and ultimate thickness. Polymer deposition takes place at the molecular level as the chemical, in dimer form, is vaporized under vacuum and heat to a dimeric gas. It is then pyrolized to cleave the dimer to its monomeric form, and finally it is deposited as a transparent polymer film. Coating thickness is controlled by deposition time. An approximate deposition rate is 5 microns per hour. The polymer deposition is generally uniform across the entire surface of the part being coated. In a single operation, a coating thicknesses from 0.10 micron to 76 microns can be applied to the piston. Preferably, a thickness of between 0.25 and 1.0 microns is applied to the piston 24. The pistons 24 are then preferably sterilized by steam before being introduced into the interior of the barrel 14.

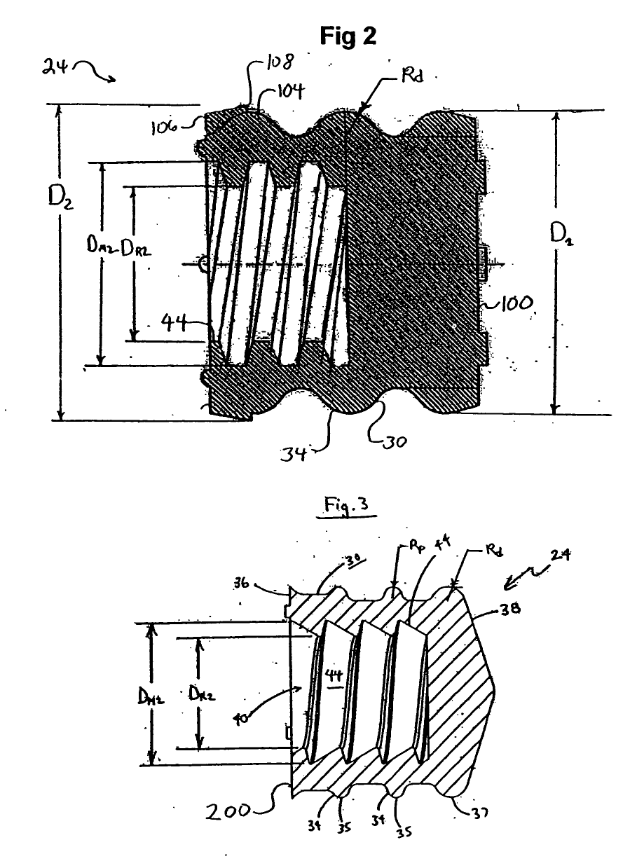

Referring to FIG. 2 in conjunction with FIG. 1, an embodiment of a geometrical design of a known prior art piston 100 is illustrated. In reference to a syringe barrel 14 of a 3 mL syringe defining an internal diameter of 8.75 mm, the piston 100 includes 2 distal lobes 102 defining a radii (Rd) of 1 mm and a diameter (D1) of 9.1 mm. The proximal lobe 104 also includes a frustoconical annular flange 106 defining a diameter (D2) at its distal edge 108 of 9.5 mm.

Referring also to FIG. 1, breakaway testing has-been conducted to determine the breakaway force for the piston 100 or various sizes inserted into a COC (Topaz) syringe barrel 14 defining the indicated volume. In each instance the barrel 14 has been e-beam sterilized. Testing has been conducted separately on pistons 100 which have been coated with parylene, and pistons 100 which have been coated with silicone. The pistons 100 were sterilized as indicated below, either by gamma sterilization or steam sterilization, and inserted into the syringe barrels 14. The time parameter in the charts below indicates the number of weeks of shelf life of the product prior to testing. The breakaway force values are provided in pounds (lbs.). Testing was conducted on an Instron testing machine according to a modified ISO standard 7886-1 Annex G “Test Method for Forces Required to Operate Plunger”. The rate of compression of the testing machine was 4 in./min.

| | | CHART 1 | |
|---|---|---|---|
| | | Gamma Sterilized Pistons | |
| | | Parylene Coating | Silicone Coating |
| | Time (T) | Avg. Force Fₛ (lbs.) | Avg. Force Fₛ (lbs.) |
| 1 mL. | T = 0 weeks | 0.90 | 0.80 |
| syringe | T = 2 weeks | 2.10 | 1.80 |
| barrel | T = 4 weeks | 2.10 | 2.50 |
| | T = 12 | 2.40 | 3.00 |
| | weeks | | |

| | | CHART 2 | |
|---|---|---|---|
| | | Steam Sterilized of Piston | |
| | | Parylene Coating | Silicone Coating |
| | Time (T) | Avg. Force Fₛ (lbs.) | Avg. Force Fₛ (lbs.) |
| 1 mL. | T = 0 weeks | 0.80 | 0.90 |
| syringe | T = 2 weeks | 1.40 | 1.80 |
| barrel | T = 4 weeks | 1.60 | 2.20 |
| | T = 12 | 1.80 | 2.40 |
| | weeks | | |

| | | CHART 3 | |
|---|---|---|---|
| | | Steam Sterilized of Piston | |
| | | Parylene Coating | Silicone Coating |
| | Time (T) | Avg. Force Fₛ (lbs.) | Avg. Force Fₛ (lbs.) |
| 3 mL | T = 0 weeks | 1.5 | 2.2 |
| syringe | T = 8 weeks | 1.9 | 4.0 |
| barrel | T = 12 | 1.7 | 3.95 |
| | weeks | | |
| | T = 26 | 2.1 | Not Available |
| | weeks | | |

| | | CHART 4 | |
|---|---|---|---|
| | | Gamma Sterilized of Piston | |
| | | Parylene Coating | Silicone Coating |
| | Time (T) | Avg. Force Fₛ (lbs.) | Avg. Force Fₛ (lbs.) |
| 10 mL | T = 0 weeks | 1.95 | 1.50 |
| syringe | T = 2 weeks | 4.60 | 5.65 |
| barrel | T = 4 weeks | 4.50 | 6.90 |
| | T = 12 weeks | 5.40 | 7.05 |

| | | CHART 5 | |
|---|---|---|---|
| | | Steam Sterilized of Piston | |
| | | Parylene Coating | Silicone Coating |
| | Time (T) | Avg. Force Fₛ (lbs.) | Avg. Force Fₛ (lbs.) |
| 10 mL | T = 0 weeks | 1.60 | 1.80 |
| syringe | T = 2 weeks | 2.40 | 5.00 |
| barrel | T = 4 weeks | 2.80 | 5.80 |
| | T = 12 weeks | 2.90 | 5.95 |

As shown in the above Charts 1-5, as the shelf life of the syringe 10 having the piston 100 increases, for syringes having e-beam sterilized barrels 14 the force required to break the piston 24 loose from its starting position is significantly less for pistons 100 having a parylene coating as opposed to pistons having a silicone coating. On average, the decrease in break away force is decreased for parylene coated pistons for all shelf life time periods of 2 weeks or more. Accordingly, it has been shown to be extremely beneficial to coat an elastomeric piston, that is located in an e-beam sterilized COC syringe barrel, with a parylene coating.

During use, the piston 24 of the present invention is located within the cavity 16 of the syringe barrel 14, as shown in FIG. 1. Referring to FIGS. 1, and 2-3 the piston 24 has an outer surface 30 which engages the inner surface 32 of the syringe barrel 14. In a preferred embodiment, the piston 24 also has a plurality of ribs or annular lobes 34 which extend radially outward from the outer surface 30 of the piston 24, and which provide a secure seal between the piston 24 and the inner surface 32 of the syringe barrel 14. Additionally, it is preferred that the geometry of the ribs 34 is configured such that (a) the piston assembly 12 is capable of being slidably displaced within the syringe barrel 14 without requiring undesirably excessive forces, and (b) the piston 24 is capable of maintaining a secure seal with the inner surface 32 of the syringe barrel 14 over an extended period of time, possibly including extended years of shelf-life. It has been found that the material, coating and geometry of the piston, as well as the mating syringe body, contribute to the contact, breakaway and sliding forces.

The piston 24 also has a proximal end 36 and a distal end 38. When the piston 24 is fitted in the syringe 10, the distal end 38 of the piston 24 is adjacent the fluid in the syringe 10. Additionally, a cavity 40 extends into the piston 24 at the proximal end 36 thereof. As is explained in greater detail below, a first mating member 44 is located in the cavity 40 of the piston 24.

As shown in FIGS. 2-5, the piston 24 may comprise a variety of geometric shapes to accomplish a variety of results. For example, the annular ridges or lobes 34 on the piston 24 may have a diameter and a radius 35 at an edge thereof which can be varied depending on the material, coating and force characteristics of the piston 24 and the syringe barrel 14. According to one aspect of the present invention, the lobes 34, and specifically the radius 35, diameter and number of lobes may be adjusted and adapted to deform against the inner surface 32 of the syringe barrel 14 to provide a positive seal therewith while maintaining acceptable breakaway and operation forces.

In the embodiments illustrated in FIGS. 2-5, each of the plurality of adjacent annular lobes 34 extend radially outward from a centerline of the piston 24. The annular lobes 34 define discrete contact areas of the piston 24 to engage the inner surface 32 of the syringe barrel 14. This modified contact area of the piston 24 decreases the surface contact area, as well as the breakaway and operational forces, between the inner surface 32 of the syringe barrel 14 and the piston 24.

Additionally, each contact area between the piston 24 and the syringe barrel 14 provides an independent seal area to maintain a sterile barrier for the contents in the fluid cavity 16 of the syringe 10. When a plurality of annular lobes 34 are utilized, a plurality of independent and distinct seal areas are formed. While three adjacent annular ridges or lobes 34 are utilized in the preferred embodiment, it is understood that the use of one or more annular lobes is acceptable without departing from the spirit of the invention.

In describing the alternate preferred embodiments of the piston 24, the description is in reference to the piston in the uncompressed state of before being inserted into a syringe barrel 14 and compressingly deformed by the inner surface 32 of the barrel.

It has been found that altering the geometry of the piston 24 lowers the breakaway forces while still utilizing a silicone coating on the piston. Referring particularly to FIGS. 3, it has been found that to alter the relative geometry of the lobes improves the ability of the piston 24 to provide a sterile seal while utilizing a silicone coating but without generating excessive breakaway forces. In the preferred embodiment a piston 200 is provided with the annular lobes 37, 34 with varying geometries. In a preferred embodiment and in reference to a syringe barrel 14 of a 3 mL syringe defining an internal diameter of 8.75 mm, the annular lobe 37 adjacent the distal end 38 of the piston 24 defines a radius (Rd) that is greater than the radius (Rp) of at least one annular nobe 34 and preferable two annular lobes disposed along the body of the piston 24 which are spaced in the proximal direction along the piston. In one preferred embodiment shown in FIG. 3, the radius (Rp) of the two annular lobes 34 along the body of the piston is approximately 0.375 mm, while the radius (Rd) of the annular nobe 37 adjacent the distal end 38 of the piston is approximately 0.750 mm. The decreased radius 35 of the annular lobes 34 along the body of the piston 24 results in decreased surface contact between the piston and the syringe barrel, and decreased breakaway forces of the piston. To provide additional assistance in decreasing the breakaway forces of the piston the minor diameter of the piston 24 defined at the base of the gap between the annular lobes 34 may be decreased.

Referring to FIGS. 2 and 3, to illustrate the reduction in breakaway force, the breakaway force for piston 100 with a silicone coating was compared with the breakaway force for piston 200 with a silicone coating over various periods of time with the following results

| | | CHART 6 | |
|---|---|---|---|
| | | Steam Sterilized/Siliconized Piston | |
| | | Piston 100 | Piston 200 |
| | Time (T) | Avg. Force Fₛ (lbs.) | Avg. Force Fₛ (lbs.) |
| 3 mL. | T = 0 | 2.3 | 0.80 |
| syringe | weeks | | |
| barrel | T = 4 | 4.0 | 1.5 |
| | weeks | | |
| | T = 12 | 3.9 | 1.9 |
| | weeks | | |
| | T = 26 | Not Available | 2.0 |
| | weeks | | |

As is illustrated, altering the geometry of the piston 200 provides a piston with significantly lower breakaway forces than the prior art piston 100.

Referring back to FIG. 2, although reducing breakaway forces, it has been found that utilizing a parylene coating on the prior art piston 100 may impact the ability of the piston 24 to form an acceptable sterile seal with the syringe barrel 14. Simply enlarging the diameter of the piston 24 to increase the pressure exerted by the piston on the barrel 14 may promote better sealing but this also leads to increased breakaway and operational forces.

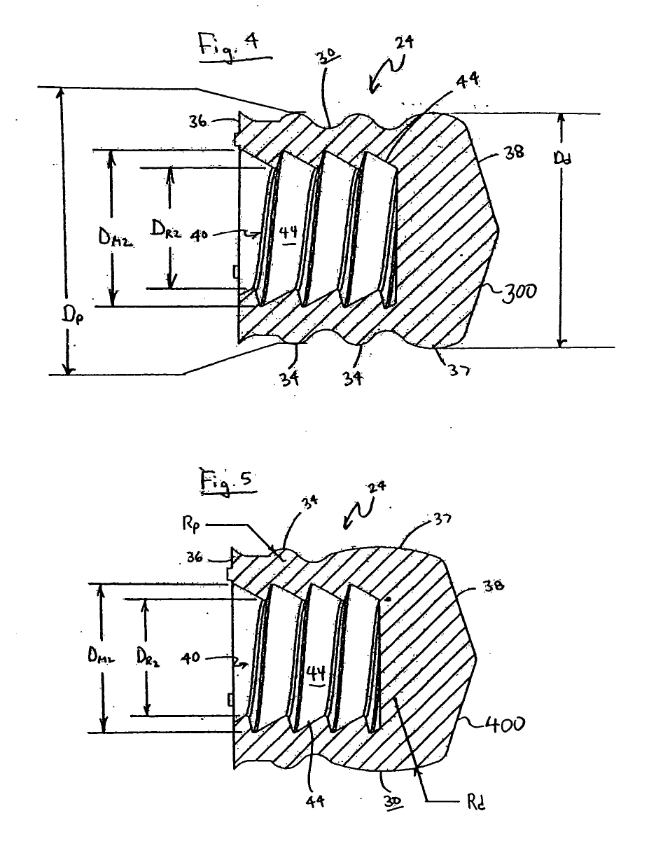

Referring to FIG. 4, in an alternate embodiment, a piston 300 is illustrated. In the uncompressed state, the outer diameter (Dp) defined by the two annular proximal lobes 34 along the body of the piston 24 may be smaller than the diameter (Dd) defined by the annular distal lobe 37 adjacent the distal end 38 of the piston 24. The radii defined by the proximal and distal lobes 34, 37 are preferably generally equal. The increase in diameter of a single lobe 37 promotes sealing but does not increase the breakaway and operational forces to an unacceptable level. As an example and in reference to a syringe barrel 14 of a 3 mL syringe defining an internal diameter of 8.75 mm, the diameter Dd defined by the distal lobe 37 is 9.35 mm and the diameter Dp of the proximal lobes 34 is 9.25 mm.

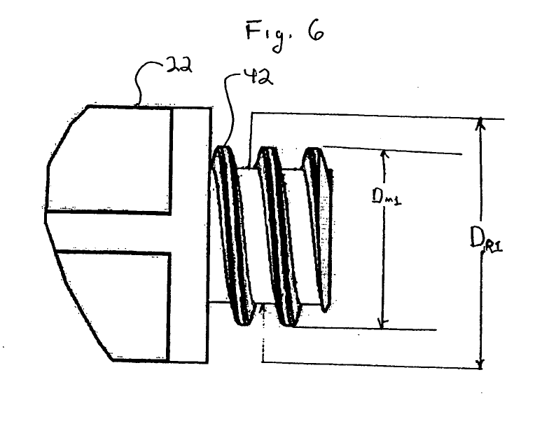

Referring to FIG. 5, in a further alternate embodiment a piston 400 is illustrated. For the piston 400 in the uncompressed state, the piston 24 includes two annular lobes 37, 34 along the surface. The first and distal annular nobe 37 adjacent the distal end 38 defines a radius of curvature which is much greater than the radius of curvature of the second and proximal lobe 34. In an example and in reference to a syringe barrel 14 of a 3 mL syringe defining an internal diameter of 8.75 mm, the radius of curvature (Rd) of the distal lobe 37 is 6.525 ml and the radius of curvature (Rp) of proximal lobe 34 is 0.750 mm.

To determine the effect of the geometries of piston 300 and piston 400 with parylene coating on the breakaway force, a breakaway study was conducted with the following results:

| | | CHART 7 | |
|---|---|---|---|
| | | Steam Sterilized/Parylene Coated Piston | |
| | | Piston 100 | Piston 200 |
| | Time (T) | Avg. Force Fₛ (lbs.) | Avg. Force Fₛ (lbs.) |
| 3 mL. | T = 0 | 21.7 | 2.0 |
| syringe | weeks | | |
| barrel | T = 4 | 2.2 | 2.7 |
| | weeks | | |
| | T = 12 | 2.3 | 2.55 |
| | weeks | | |
| | T = 26 | 2.2 | 2.6 |
| | weeks | | |

As can be seen, piston 300 and piston 400 exhibit low breakaway forces with the parylene coatings.

To determine the sealing performance of the various embodiments, the pistons were subjected to two standard syringe test to determine their sealing performance. The first test is and axial test ISO 7886-1 Annex D “Test Method for Liquid Leakage of Syringe Piston under Compression” which was performed at 90 psi and the second test is a vacuum test ISO 7886-1Annex B “Test Method for Air Leakage Past Syringe Piston during Aspiration” which was conducted at 88 kPa of pressure below ambient air pressure. The axial test would determine if liquid would migrate through the lobes with a failure occurring if the liquid passed around all three lobes. The vacuum test would determine if air migrated around the lobes with a failure occurring if any air migrated around the lobes.

CHART 8

| | Axial Test | | ISO Vacuum |
|---|---|---|---|
| Group | Failures | Liquid in Lobes | Failures |
| Piston 100 - Siliconized | 0/40 | 0/40 | 0/75 |
| Piston 200 - Siliconized | 0/40 | 0/40 | N/A |
| Piston 100 - Parylene | 0/10 | 1/10 | 5/10 |
| Piston 300 - Parylene | 0/10 | 0/10 | 0/10 |
| Piston 400 - Parylene | 0/10 | 0/10 | 0/10 |

As the results show, Piston 100 with a parylene coating exhibited failures but Piston 300 and 400, which had just slightly greater breakaway forces, showed no failures.

Referring back to FIG. 1, like the syringe bodies 14, the components of the piston assembly 12, and specifically the piston tip 24, are typically manufactured from various polymeric materials, including various elastomers. Preferably, the components of the piston assembly 12 used in conjunction with the COCs set forth above are fabricated from a polymeric material and more preferably a polymeric material that will not generate unacceptable levels of halogens after processing, filling with fluid for dispensing, sterilization and storage. Suitable polymeric materials include synthetic rubbers including styrene-butadiene copolymer, acrylonitrile-butadiene copolymer, neoprene, butyl rubber, polysulfide elastomer, urethane rubbers, stereo rubbers, ethylene-propylene elastomers. In a preferred form of the invention, a halogenated butyl rubber and more preferably a chlorobutyl-based elastomer is utilized.

Typically, the piston assembly 12 is assembled by connecting the plunger rod 22 with the piston 24 (more particularly shown in FIGS. 2-5). In a preferred embodiment, the plunger rod 22 is connected to the piston 24 in a threaded engagement. Referring also to FIG. 6, to accomplish such a connection, the piston 24 has a first mating member 44 and the plunger rod 22 has a second mating member 42 which threadedly engages the first mating member. In the most preferred embodiments, the plunger rod 22 has a plurality of male threads 42 which comprise the second mating member 42, and the piston 24 has a plurality of female threads 44 which comprise the first mating member 6. During connection, the male threads 42 of the plunger rod 22 engage the female threads 44 of the piston 24 in a mating engagement to removably secure the plunger rod 22 to the piston 24.

As shown in FIG. 6, the male threads 42 of the plunger rod 22 of the present invention have a major diameter (D_{M1}) and a minor diameter (D_{R1}). Similarly, as shown in FIGS. 3-6, the female threads 44 of the piston 24 of the present invention have a major diameter (D_{M2}) and a minor diameter (D_{R2}). In prior art embodiments, the major diameter (D_{M1}) of the male threads of the plunger rod was equal to the major diameter (D_{M2}) of the female threads of the piston, and the minor diameter (D_{R1}) of the male threads of the plunger rod was equal to the minor diameter (D_{R2}) of the female threads of the piston. Additionally, the thread pitch of the prior art piston was equal to the thread pitch of the prior art plunger rods. It was previously thought that having mating components with the same dimensional thread characteristics provided a secure connection when properly fitted together.

In known plunger rods and pistons, the major diameter of the threads 44 of the piston 24 is nominally larger than the major diameter of the male threads 42 of the plunger rod 22. For example and referring to FIG. 2, in a known piston for a 3 mL syringe, the major diameter (D_{M2}) of the threads 44 of the piston is 6.2 mm which is slightly larger than major diameter (D_{M1}) of the male threads 42 which have a major diameter of 6 mm. Similarly the minor diameter (D_{R1}) of the threads 44 of the piston 24 is nominally larger than the minor diameter (D_{R2}) of the male threads 42 of the plunger rod 22. For example in a known piston for a 3 mL syringe, the minor diameter of the threads 44 of the piston is 4.7 mm which is slightly larger than major diameter of the male threads 42 which have a major diameter of 4.5 mm.

It has been discovered that when plunger rods and mating pistons having nominally differing dimensional thread characteristics are connected during and automated assembly process, there is an unacceptably high incidence of unacceptable rod 22 and piston 24 attachment.

For example it has been found that in the known piston 24 and rod 22 attachment, in automatic insertion equipment (not shown) misthreading of the rod 22 into the piston 24 occurred in more than 7% of the attachments and incomplete threading (where the piston has a tendency to spin within the syringe barrel 14 prior to the plunger rod 22 being fully seated within the piston) occurred in more than 7% of the attachments. Experiments have uncovered that spinning of the piston within the syringe barrel may result in the breaking of the sterile. barrier. An additional shortcoming of the piston spinning in the syringe barrel prior to be fully seated on the plunger rod is that the components of the piston assembly are not fully secured to one another.

Accordingly, in the present invention, the dimensional characteristics of one of the first mating member 44 of the piston, or the second mating member 42 of the plunger rod 22 has been slightly decreased. Conversely, the dimensional characteristics of one of the first mating member 44 of the piston, or the second mating member 42 of the plunger rod 22 may be slightly increased. Alternatively, one of the first mating member 44 of the piston, or the second mating member 42 of the plunger rod 22 may be slightly decreased in size, while the other of the first mating member 44 of the piston, or the second mating member 42 of the plunger rod 22 may be slightly increased in size.

In a preferred embodiment, the male threads 42 of the plunger rod 22 have been decreased in size, while the dimensional characteristics female threads 44 of the piston 24 have remained constant. According to this modification, the major diameter (D_{M1}) of the threads of the plunger rod 22 is even less than the major diameter (D_{M2}) of the threads of the piston 24 in the known pistons and rods. Similarly, the minor diameter (D_{R1}) of the threads of the plunger rod 22 is made to be even less than the minor diameter (D_{R2}) of the threads of the piston 24. The thread pitch of the threads of the piston 24 and the plunger rod 22 has remained the same. In a preferred embodiment of a 3 ml. syringe, the female threads 44 of the piston 24 have a major diameter (D_{M2}) of 6.2 mm., and a minor diameter (D_{R2}) of 4.7 mm, while the male threads 42 of the plunger rod 22 have been decreased in size and have a major diameter (D_{M1}) of 5.54 mm., and a minor diameter (D_{R1}) of 4.2 mm. By decreasing the thread size of the major and minor diameters of the threads of the plunger rod, the interference between the plunger rod and the piston has been decreased. In the automated assembly equipment misthreading of the rod 22 into the piston 24 occurred in less than 1% of the attachments and incomplete threading (where the piston has a tendency to spin within the syringe barrel 14 prior to the plunger rod 22 being fully seated within the piston) occurred in less than 7% of the attachments

Referring back to FIG. 1, one method of sterilizing, filling and assembly for the syringes 10 is described below. First, the syringe body 14 and attached tip cap 21 is sterilized at a sterilization station. This may include a terminal process performed within an autoclave or an irradiation process. If performed in an autoclave, the sterilization medium is typically steam. Gamma radiation may also provided to sterilize the syringe bodies through irradiation. In the preferred methods of the present invention, however, electron beam (e-beam) irradiation is provided to sterilize the syringe bodies. E-beam irradiation is preferable to steam because irradiation sterilization is faster, it saves manufacturing space, and steam creates waste and causes a material handling problem. E-beam irradiation is preferable over gamma radiation because e-beam irradiation is less damaging to the syringe bodies and it is faster. With e-beam irradiation, there is less coloration of the polymeric material; thus, the clinician's ability to inspect the syringe body and its contents is improved.

The e-beam dose delivered to the syringe bodies is preferably in the range of 10-50 kGy, or any range or combination of ranges therein, and more preferably 25 kGy at approximately 1 MeV to 10 MeV, or any range or combination of ranges therein, but preferably less than or equal to 1 MeV. Once individual syringe bodies are sterilized, they are sterile transferred to a sterile environment to maintain the sterility of the syringe bodies.

The pistons 24 are manufactured and preferably coated with a parylene coating. Next, the pistons 24 are sterilized in any conventional manner but are preferably processed through a gamma radiation or steam. The sterilized piston 24 is transferred into the sterile environment

The next step includes at least three sub-steps, namely filling the syringe bodies with a sterile medical solution and adding the piston to an open end of each syringe body. The medical solution is filled into the syringe bodies via the open end of the syringe bodies that is opposite the end having the tip cap 21. The medical solution can also be introduced through the tip end without departing from the spirit of the invention.

Once filled with the medical solution, the step of inserting a piston into the open end of each syringe body 14 is carried out. The piston 24 forms a seal with an inner sidewall 32 of the syringe body 14, and the medical solution is sealed within the syringe body. The pistons 24 may be automatically added to the syringe bodies as part of the filler process.

The next step is transferring the syringe bodies to the packaging station. At the automated packaging station a plunger rod 22 is fixedly attached to the piston 24 via mating engagement described above, and the finished syringes are inspected, labeled, and packaged for shipment to an end user. It is contemplated that no human intervention is required to inspect, label, and package the syringe bodies.

It will be understood that the invention may be embodied in other specific forms without departing from the spirit or central characteristics thereof. The present embodiments, therefore, are to be considered in all respects as illustrative and not restrictive, and the invention is not to be limited to the details given herein.

1. A syringe assembly comprising:
a barrel of one of a cyclic olefin containing polymer anda bridged polycyclic olefin containing polymer, the barrel including an inner surface defining a chamber to contain flowable materials, the chamber having an opening; and,
an elastomeric piston slidably attached to the body and providing a seal of the chamber, the elastomeric piston having a parylene coating on an outer surface thereof.

2. The assembly of claim 1, wherein the elastomeric piston is steam sterilized.

3. The assembly of claim 1, wherein the elastomeric component is a synthetic rubber selected from the group consisting of styrene-butadiene copolymers, acrylonitrile-butadiene copolymers, neoprenes, butyl rubbers, polysulfide elastomers, urethane rubbers, stereo rubbers, ethylene-propylene elastomers.

4. The assembly of claim 3, wherein the synthetic rubber is a halogenated butyl rubber.

5. The assembly of claim 1, wherein the body is e-beam sterilized.

6. The assembly of claim 1, wherein the piston has a plurality of annular lobes providing multiple seal areas with said inner surface of said barrel.

7. The assembly of claim 6, wherein the annular lobe adjacent a distal end of the piston has a radius which is greater than a radius of the annular lobe located adjacent a proximal end of the piston.

8. The assembly of claim 1, further comprising a plunger rod having a first mating member which engages a second mating member of the piston to removably connect the plunger rod to the piston.

9. The assembly of claim 8, wherein the first mating member of the plunger rod has a series of threads having a major diameter and a minor diameter, wherein the second mating member has a series of threads having a major diameter and a minor diameter, and wherein the major and minor diameters of one of the first and second mating members are smaller than the major and minor diameters of the other of the first and second mating members.

10. The assembly of claim 9, wherein a thread pitch for the first mating member is the same as the thread pitch for the second mating member.

11. The assembly of claim 6, wherein the annular lobe adjacent a distal end of the piston has a diameter which is greater than a diameter of the annular lobe located adjacent a proximal end of the piston.

12. A piston assembly for use in a flowable materials container comprising:
a piston having a proximal end, a distal end, and a cavity extending into the piston at the proximal end thereof, wherein a first mating member is located in the cavity, and wherein the first mating member comprises one of a plurality of male and female threads,
a plunger rod having a second mating member comprising the other of the plurality of male and female threads, wherein the first mating member engages the second mating member, and wherein a major diameter and a minor diameter of one of the first and second mating members is larger than the major diameter and minor diameter of the other of the first and second mating members.

13. The piston assembly of claim 12, wherein the first mating member and the second mating member have the same thread pitch.

14. The piston assembly of claim 12, further comprising a plurality of annular ribs extending radially outward from an outer surface of the piston.

15. The piston assembly of claim 12, wherein at least one of the annular ribs has a radius at an edge thereof which is smaller than the radius of another of the annular ribs.

16. The piston assembly of claim 12, wherein the piston is coated with a parylene coating.

17. The piston assembly of claim 12, wherein the piston is made of an elastomeric material.

18. The piston assembly of claim 17, wherein the piston is made of a halogenated butyl rubber.

19. The piston assembly of claim 18, wherein the piston assembly is positioned in a syringe body made of a cyclic olefin containing polymer or a bridged polycyclic olefin containing polymer.
